(19) United States
(12) Patent Application Publication
(10) Pub. No.: US 2012/0283245 A1
(43) Pub. Date: Nov. 8, 2012

(54) AZABICYCLO[3.1.0]HEX-2-YL COMPOUNDS, A PROCESS FOR THEIR PREPARATION AND PHARMACEUTICAL COMPOSITIONS CONTAINING THEM

(75) Inventors: Patrick Casara, Perpignan (FR); Anne-Marie Chollet, Le Pecq (FR); Alain Dhainaut, Chatou (FR); Jean-Michel Henlin, Suresnes (FR); Pierre Lestage, La Celle Saint Cloud (FR); Fany Panayi, Paris (FR)

(73) Assignee: LES LABORATOIRES SERVIER, SURESNES CEDEX (FR)

(21) Appl. No.: 13/514,796

(22) PCT Filed: Dec. 8, 2010
(86) PCT No.: PCT/FR2010/000823

§ 371 Date: Jul. 16, 2012

(30) Foreign Application Priority Data
Dec. 9, 2009 (FR) 09.05953

(51) Int. Cl. A61K 31/403; A61K 31/445; A61K 31/55; A61P 25/00; A61P 29/00; A61P 25/28; A61P 25/16; A61P 25/20; A61P 25/22; A61P 25/24; C07D 209/52; A61P 25/18

(52) U.S. Cl. 514/215; 548/452; 514/412; 514/319

(57) ABSTRACT

Compounds of formula:

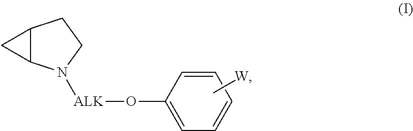

wherein:
ALK represents an alkylene chain,
W represents a group

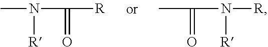

wherein R and R′ are as defined in the description.

Medicinal products containing the same which are useful in treating cognitive disorders associated with cerebral aging or with neurodegenerative diseases.

The present invention relates to new azabicyclo[3.1.0]hex-2-yl compounds, to a process for their preparation and to pharmaceutical compositions containing them.

The compounds of the present invention are especially valuable from a pharmacological point of view for their interaction with central histaminergic systems in vivo.

Ageing of the population due to increased life expectancy at birth has brought with it a large increase in the incidence of age-related neuropathologies and especially of Alzheimer's disease. The principal clinical manifestations of cerebral ageing and especially of age-related neuropathologies are deficiencies in memory and cognitive functions, which may lead to dementia.

Neuropharmacological studies have shown that, in the central nervous system, histamine, via the central histaminergic systems, has the role of a neurotransmitter or neuromodulator in physiological or physiopathological situations (Pell and Green, Annu. Rev. Neurosci., 1986, 9, 209-254; Schwartz et al., Physiol. Rev., 1991, 71, 1-51). Thus, it has been shown that histamine is involved in various physiological and behavioural processes, such as thermoregulation, neuro-endocrinal regulation, nociception, circadian rhythm, cataleptic states, motility, aggressiveness, eating behaviour, learning and memorisation, and synaptic plasticity (Hass et al., Histaminergic neurones:morphology and function, Boca Raton, Fla.:CRC Press, 1991, pp. 196-208; Brown et al., Prog. Neurobiology, 2001, 63, 637-672; Smith et al., Neuroimmunomodulation 2007, 14, pp. 317-325).

Studies carried out in animals have shown that an increase in endogenous extra-synaptic levels of histamine makes it possible to promote states of vigilance, learning and memory processes, and to regulate food intake (Brown et al., Prog. Neurobiol., 2000, 63, 637-672; Passani et al., Neurosci. Biobehay. Rev., 2000, 24, 107-113). As a result, the potential therapeutic indications for compounds capable of increasing the turnover or release of histamine at the central level are the treatment of cognitive deficiencies associated with cerebral ageing, with acute and chronic neurodegenerative diseases and with schizophrenia and also the treatment of mood disorders, of Tourette's syndrome (Gulhan Ercan-Sencicek et al., New England Journal of Medicine, May 20, 2010, 1901-1908), of schizophrenia, of sleep disorders, of sleep-waking rhythm disorders and of attention-deficit hyperactivity syndrome. Furthermore, studies have shown that an injection of histamine into the central hypothalamic nuclei involved in the regulation of satiety attenuates feeding in the rat. Hypofunctioning of histaminergic transmission has moreover been demonstrated in genetically obese rats (Machidori et al., Brain Research, 1992, 590, 180-186). Consequently, eating behaviour disorders and obesity are also potential therapeutic indications for the compounds of the present invention.

The present invention relates to new azabicyclic compounds which are distinguished from the compounds mentioned in Application WO2005/089747 by the presence of a 2-azabicyclo[3.1.0]hexane ring system.

At the neurological level, these new compounds open the way not only to new treatments for cognitive disorders associated with cerebral ageing, with neurodegenerative diseases or with cranial traumas but also to the treatment of psycho-behavioural disorders associated with those pathologies, such as sleep disorders, apathy and/or depressive states. The pharmacological profile of the compounds of the invention moreover also makes it possible to envisage new treatments in the psychiatric field, for example for Tourette's syndrome, schizophrenia, mood disorders or sleep disorders.

The present invention relates, more specifically, to compounds of formula (I):

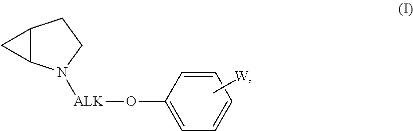

wherein:
ALK represents an alkylene chain,
W represents a group

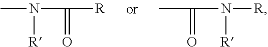

in which R and R′, each independently of the other, represent a hydrogen atom or a linear or branched (C₁-C₆)alkyl group optionally substituted by one or more groups selected from halogen, hydroxy and alkoxy,

it being understood that:
the term “alkylene” denotes a linear or branched divalent radical containing from 2 to 6 carbon atoms,
the term “alkoxy” denotes an alkyl-oxy group in which the alkyl chain, which is linear or branched, contains from 1 to 6 carbon atoms,

to their enantiomers and diastereoisomers, and also to addition salts thereof with a pharmaceutically acceptable acid or base.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, Cert-butylamine etc.

Compounds of formula (I) to which preference is given are those wherein the W group is located in the para position.

ALK preferably represents a linear divalent radical containing from 2 to 6 carbon atoms such as, for example, an ethylene or propylene group, more preferably still a propylene group.

A particular embodiment of the invention relates to compounds of formula (I) wherein W represents the group

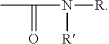

Another particular embodiment of the invention relates to compounds of formula (I) wherein W represents the group

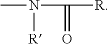

Advantageously, R and R′, each independently of the other, represent a hydrogen atom, a methyl group or an ethyl group, those groups optionally being substituted by a methoxy group.

More especially, W represents a group —CO—NH—CH₃, —CO—N(CH₃)₂, —CO—NH₂, —CO—N(CH₂CH₃)₂, —NH—CO—CH₃, —N(CH₃)—CO—CH₃ or —NH—CO—CH₂—OCH₃.

Even more especially, the invention relates to the compounds of formula (I) which are:
4-{3-(cis-2-azabicyclo[3.1.0]hex-2-yl)propoxy}-N,N-dimethylbenzamide,
4-{3-(cis-2-azabicyclo[3.1.0]hex-2-yl)propoxy}-N,N-diethylbenzamide,
N-(4-{3-(cis-2-azabicyclo[3.1.0]hex-2-yl)propoxy}phenyl)-N-methylacetamide,
4-[3-(cis-2-azabicyclo[3.1.0]hex-2-yl)propoxy]benzamide,
N-(4-{3-(cis-2-azabicyclo[3.1.0]hex-2-yl)propoxy}phenyl)acetamide,
4-{3-(cis-2-azabicyclo[3.1.0]hex-2-yl)propoxy}-N-methylbenzamide,
N-(4-{3-(cis-2-azabicyclo[3.1.0]hex-2-yl)propoxy}phenyl)-2-methoxyacetamide,
N-(4-{2-(cis-2-azabicyclo[3.1.0]hex-2-yl)ethoxy}phenyl)acetamide,

and their enantiomers, and also addition salts thereof with a pharmaceutically acceptable acid or base.

Among the addition salts with a pharmaceutically acceptable acid, preference is given more especially to hydrochlorides, oxalates and citrates.

The invention relates also to a process for the preparation of compounds of formula (I), which process is characterised in that there is used as starting material the compound of formula (II):

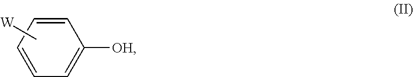

wherein W is as defined for formula (I),

with which compound of formula (II) there is condensed, in a basic medium, the compound of formula (III):

Br-ALK-Cl (III),

wherein ALK is as defined for formula (I),

to obtain the compound of formula (IV):

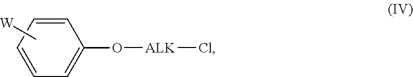

wherein W and ALK are as defined hereinbefore,

with which there is condensed the compound of formula (V):

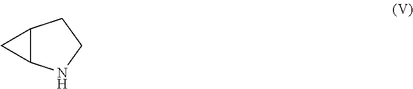

to yield the compound of formula (I) as defined hereinbefore:

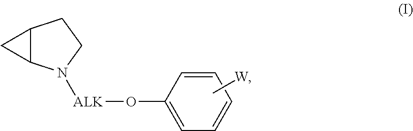

which may be purified according to a conventional separation technique, is converted, if desired, into its addition salts with a pharmaceutically acceptable acid or base and is separated, where appropriate, into its optical isomers according to a conventional separation technique.

The compounds of formulae (II), (III) and (V) are either commercially available or can be obtained by the person skilled in the art using conventional chemical reactions described in the literature.

Alternatively, compounds of formula (VI):

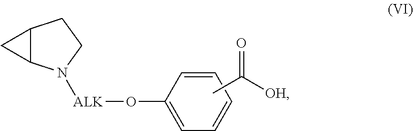

wherein the ALK group is as defined hereinbefore,

can be used as synthesis intermediates for compounds of formula (I/a), particular cases of compounds of formula (I), wherein W represents a —CONRR′ group, by coupling with an amine of formula NHRR′, wherein R and R′ are as defined for formula (I).

Similarly, compounds of formula (VII):

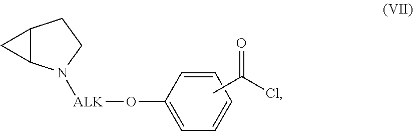

wherein the ALK group is as defined hereinbefore,

can be used as synthesis intermediates for compounds of formula (I/a), particular cases of compounds of formula (I), wherein W represents a —CONRR′ group, by coupling with an amine of formula NHRR′, wherein R and R′ are as defined for formula (I).

Furthermore, compounds of formula (I/a), particular cases of compounds of formula (I), wherein W represents a —CONRR′ group, may also be obtained by condensation of the amine NHRR′, wherein R and R′ are as defined for formula (I), using compounds of formula (VIII):

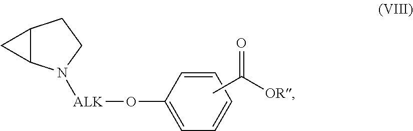

wherein the ALK group is as defined hereinbefore and R″ represents a linear or branched (C₁-C₆)alkyl group or a benzyl group,

the compounds of formula (VIII) being prepared via the corresponding carboxylic acid (VI) or acyl chloride (VII) shown hereinbefore.

Finally, it is also possible to obtain compounds of formula (I/a) by hydrolysing compounds of formula (IX):

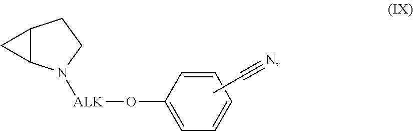

wherein the ALK group is as defined hereinbefore.

At the neurological level, the compounds according to the invention may be useful in the treatment of cognitive disorders associated with cerebral ageing or with neurodegenerative diseases such as Alzheimer's disease, Parkinson's disease, Pick's disease, Lewy body dementias, frontal and subcortical dementias, frontotemporal dementias, vascular dementias, Huntington's disease and multiple sclerosis, in new treatments for cognitive disorders associated with cranial traumas, but also in the treatment of psycho-behavioural disorders associated with those pathologies, such as sleep disorders, apathy and anxio-depressive states. Sleep disorders associated with Alzheimer's disease and with Parkinson's disease, such as diurnal hypersomnolence, especially are targets.

At the psychiatric level, these compounds may be useful in the treatment of mood disorders, and more especially in the treatment of anxio-depressive states, of Tourette's syndrome, of schizophrenia and of cognitive disorders associated therewith, and of pain, and also in the treatment of sleep disorders, of sleep-waking rhythm disorders and of attention-deficit hyperactivity syndrome (ADHD). Among the sleep disorders there may be more especially mentioned narcolepsy and sleep apnoea. Sleep disorders such as hypersomnia occurring in obstructive sleep apnoea syndrome or in attention-deficit hyperactivity syndrome, and also diurnal somnolence are also targets.

The present invention relates also to pharmaceutical compositions comprising one compound of formula (I) in combination with one or more pharmaceutically acceptable excipients.

In the pharmaceutical compositions according to the invention, the weight proportion of active ingredient (weight of the active ingredient over the total weight of the composition) is from 1 to 50%.

Among the pharmaceutical compositions according to the invention, there may be mentioned more especially those that are suitable for oral, parenteral, nasal, per- or trans-cutaneous, rectal, perlingual, ocular or respiratory administration, especially tablets or dragées, sublingual tablets, sachets, paquets, capsules, glossettes, lozenges, suppositories, creams, ointments, dermal gels, and drinkable or injectable ampoules.

The useful dosage varies according to the sex, age and weight of the patient, the administration route, the nature of the therapeutic indication, and any associated treatments, and ranges from 0.05 mg to 500 mg per 24 hours for treatment in from 1 to 3 administrations per day.

The association of a compound of formula (I) with an acetylcholinesterase inhibitor also forms an integral part of the invention, and more especially still the association of a compound of formula (I) with donepezil, rivastigmine or galantamine. Associations of this type may be used in the treatment of cognitive disorders of Alzheimer's disease.

The following Examples illustrate the invention but do not limit it in any way. The structures of the compounds described in the Examples were determined in accordance with the usual spectrophotometric techniques (infrared, NMR, mass spectrometry etc.).

By way of information, the compounds hereinbelow correspond to racemates of cis configuration; in other words, these compounds correspond to racemic mixtures of (1R,5S)-2-azabicyclo[3.1.0]hex-2-yl skeletons and (1S,5R)-2-azabicyclo[3.1.0]hex-2-yl skeletons.

As mentioned in the Examples hereinbelow, racemic mixtures may be separated, in order to obtain pure enantiomers, by chiral separation techniques on an HPLC column, for example of CHIRALCEL OF, CHIRALPACK AS-H, CHIRALPACK T304 or CHIRALPACK AD-H type.

### EXAMPLE 1

### Synthesis Route A: 4-[3-(cis-2-Azabicyclo[3.1.0]hex-2-yl)propoxy]-benzamide hydrochloride

Step 1: 4-(3-Chloropropoxy)benzamide

A mixture composed of 0.004 mol of 4-hydroxybenzamide, 0.004 mol of 1-bromo-3-chloropropane and 0.006 mol of caesium carbonate in 10 ml of acetonitrile is heated at reflux for 5 hours.

Step 2: 4-[3-(cis-2-Azabicyclo[3.1.0]hex-2-yl)propoxy]benzamide

To the reaction mixture of Step 1, at ambient temperature, there are added 0.004 mol of cis-2-azabicyclo[3.1.0]hexane, the synthesis of which is described in J. Org. Chem. 1994, 59, 276-277, and 0.002 mol of sodium iodide. Heating at reflux is then resumed for 16 hours. The precipitate is filtered off and rinsed with acetonitrile. The filtrate is concentrated to dryness. The residue is taken up in dichloromethane. The resulting solution is extracted with sodium hydroxide solution and then with water, before being dried over magnesium sulphate and concentrated to dryness. The residue is purified by a preparative chromatography technique on a Lichroprep RP-18 phase.

Step 3: 4-[3-(cis-2-Azabicyclo[3.1.0]hex-2-yl)propoxy]benzamide hydrochloride

The product obtained in Step 2 is dissolved in 10 ml of ethanol to which 2 ml of 2N ethereal HCl are added. The product thereby obtained is filtered off, rinsed with ethanol and dried in vacuo.

Elemental Microanalysis:

| | % C | % H | % N | % Cl | % Cl− |
|---|---|---|---|---|---|
| Calculated | 60.70 | 7.13 | 9.44 | 11.95 | 11.95 |
| Found | 60.44 | 7.28 | 9.47 | 12.30 | 11.75 |

### EXAMPLE 1

### Route B: 4-[3-(cis-2-Azabicyclo[3.1.0]hex-2-yl)propoxy]benzamide hydrochloride

Step 1: 4-{3-(cis-2-Azabicyclo[3.1.0]hex-2-yl)propoxy}benzonitrile

The test procedure is the same as that of Example 1, synthesis route A, Steps 1 and 2, replacing the 4-hydroxybenzamide in Step 1 by 4-hydroxybenzonitrile.

Step 2: 4-[3-(cis-2-Azabicyclo[3.1.0]hex-2-yl)propoxy]benzamide hydrochloride

The compound obtained in the Step above (2.2 g) is dissolved in 90 ml of ethanol and heated at reflux in the presence of 5.1 g of KOH for 18 hours. The mixture is poured into 90 ml of water and then concentrated to half volume in vacuo. The solid obtained is filtered off, rinsed with isopropyl ether and then dissolved in 10 ml of ethanol to which 2 ml of 2N ethereal HCl are added. The product thereby obtained is filtered off, rinsed with ethanol and dried in vacuo.

Elemental Microanalysis:

| | % C | % H | % N | % Cl | % Cl− |
|---|---|---|---|---|---|
| Calculated | 60.70 | 7.13 | 9.44 | 11.95 | 11.95 |
| Found | 60.50 | 7.20 | 9.50 | 12.45 | 12.35 |

### EXAMPLE 1

### Route C: 4-[3-(cis-2-Azabicyclo[3.1.0]hex-2-yl)propoxy]benzamide hydrochloride

Step 1: Methyl 4-[3-(cis-2-azabicyclo[3.1.0]hex-2-yl)propoxy]benzoate

The test procedure is the same as that of Example 1, synthesis route A, Steps 1 and 2, replacing the 4-hydroxybenzamide in Step 1 by methyl 4-hydroxybenzoate.

Step 2: 4-[3-(cis-2-Azabicyclo[3.1.0]hex-2-yl)propoxy]benzoic acid

A mixture of 3.5 g of the compound of Step 1, of 12.7 ml of 2N sodium hydroxide solution and 8 ml of methanol is heated at reflux for one hour. To the reaction mixture, cooled in an ice bath, there are added 12.7 ml of 2N HCl. The precipitate is washed with water and dried in vacuo.

Step 3: 4-{3-(cis-2-Azabicyclo[3.1.0]hex-2-yl)propoxy}benzoyl chloride hydrochloride

A mixture of 1.8 g of the product described in Step 2 and 20 ml of thionyl chloride is heated at reflux for 2 hours. The reaction mixture is concentrated in vacuo and co-evaporated twice with toluene. The solid residue is homogenised in ethyl ether, filtered and dried in vacuo.

Step 4: 4-[3-(cis-2-Azabicyclo[3.1.0]hex-2-yl)propoxy]benzamide hydrochloride

To a solution of 1 g of the product described in Step 3 in dichloromethane at 0° C. there are added, dropwise, 4 ml of 2N ammoniacal methanol. The mixture is then stirred for 1 hour at ambient temperature and is washed with 2N sodium hydroxide solution and then with water. The organic phase is dried over magnesium sulphate and concentrated. The solid obtained is filtered off, rinsed with isopropyl ether and then dissolved in 10 ml of ethanol to which 2 ml of 2N ethereal HCl are added. The product thereby obtained is filtered off, rinsed with ethanol and dried in vacuo.

Elemental Microanalysis:

| | % C | % H | % N | % Cl | % Cl− |
|---|---|---|---|---|---|
| Calculated | 60.70 | 7.13 | 9.44 | 11.95 | 11.95 |
| Found | 60.44 | 7.28 | 9.47 | 12.30 | 11.75 |

### EXAMPLE 2

### 4-{2-(cis-2-Azabicyclo[3.1.0]hex-2-yl)ethoxy}benzamide hydrochloride

The test procedure is the same as that of Example 1, synthesis route A, replacing the 1-bromo-3-chloropropane in Step 1 by 1-bromo-2-chloroethane.

Elemental Microanalysis:

| | % C | % H | % N | % Cl | % Cl− |
|---|---|---|---|---|---|
| Calculated | 59.47 | 6.77 | 9.91 | 12.54 | 12.54 |
| Found | 59.60 | 6.99 | 9.97 | 12.30 | 12.16 |

### EXAMPLE 3

### N-(4-{3-(cis-2-azabicyclo[3.1.0]hex-2-yl)propoxy}phenyl)acetamide hydrochloride

The test procedure is the same as that of Example 1, synthesis route A, replacing the 4-hydroxybenzamide in Step 1 by N-(4-hydroxyphenyl)acetamide.

Elemental Microanalysis:

| | % C | % H | % N | % Cl | % Cl− |
|---|---|---|---|---|---|
| Calculated | 61.83 | 7.46 | 9.01 | 11.41 | 11.41 |
| Found | 61.62 | 7.38 | 9.01 | 11.55 | 11.38 |

### EXAMPLE 4

### N-(4-{2-(cis-2-azabicyclo[3.1.0]hex-2-yl)ethoxy}phenyl)acetamide hydrochloride

The test procedure is the same as that of Example 2, replacing the 4-hydroxybenzamide in Step 1 by N-(4-hydroxyphenyl)acetamide.

Elemental Microanalysis:

| | % C | % H | % N | % Cl | % Cl− |
|---|---|---|---|---|---|
| Calculated | 60.70 | 7.13 | 9.44 | 11.95 | 11.95 |
| Found | 60.25 | 7.01 | 9.59 | 11.95 | 11.84 |

### EXAMPLE 5

### 4-{3-(cis-2-Azabicyclo[3.1.0]hex-2-yl)propoxy}-N,N-dimethylbenzamide hydrochloride

Step 1: 4-[3-(cis-2-Azabicyclo[3.1.0]hex-2-yl)propoxy]-N,N-dimethylbenzamide

The test procedure is the same as that of Example 1, synthesis route A, Steps 1 and 2, replacing the 4-hydroxybenzamide in Step 1 by 4-hydroxy-N,N-dimethylbenzamide.

Elemental Microanalysis:

| | % C | % H | % N |
|---|---|---|---|
| Calculated | 70.80 | 8.39 | 9.71 |
| Found | 69.33 | 8.47 | 9.52 |

Step 2: 4-[3-(cis-2-Azabicyclo[3.1.0]hex-2-yl)propoxy]-N,N-dimethylbenzamide hydrochloride

The test procedure is the same as that of Example 1, synthesis route A, Step 3.

### EXAMPLE 6

### 4-{3-(cis-2-Azabicyclo[3.1.0]hex-2-yl)propoxy}-N,N-diethylbenzamide hydrochloride

Step 1: 4-[3-(cis-2-Azabicyclo[3.1.0]hex-2-yl)propoxy]-N,N-diethylbenzamide

The test procedure is the same as that of Example 1, synthesis route A, Steps 1 and 2, replacing the 4-hydroxybenzamide in Step 1 by 4-hydroxy-N,N-diethylbenzamide.

Elemental Microanalysis:

| | % C | % H | % N |
|---|---|---|---|
| Calculated | 72.12 | 8.92 | 8.85 |
| Found | 71.69 | 8.72 | 8.64 |

Step 2: 4-[3-(cis-2-Azabicyclo[3.1.0]hex-2-yl)propoxy]-N,N-diethylbenzamide hydrochloride

The test procedure is the same as that of Example 1, synthesis route A, Step 3.

### EXAMPLE 6a

### 4-{3-(cis-2-Azabicyclo[3.1.0]hex-2-yl)propoxy}-N,N-diethylbenzamide hydrochloride (enantiomer 1)

Enantiomer 1 was obtained by preparative separation on a chiral column, CHIRALPACK T304 loaded at 1 g/kg, eluant mixture: acetonitrile/diethylamine (100/0.1), flow rate 100 ml/min, UV detection at 270 nm

Optical rotation: [α_{D}]_{589 nm}^{20°}=−53.79° (c=0.98; MeOH)

### EXAMPLE 6b

### 4-{3-(cis-2-Azabicyclo[3.1.0]hex-2-yl)propoxy}-N,N-diethylbenzamide hydrochloride (enantiomer 2)

Enantiomer 2 was obtained by preparative separation on a chiral column, CHIRALPACK T304 loaded at 1 g/kg, eluant mixture: acetonitrile/diethylamine (100/0.1), flow rate 100 ml/min, UV detection at 270 nm

Optical rotation: [α_{D}]_{589 nm}^{20°}=+54.02° (c=1.02; MeOH)

### EXAMPLE 7

### 4-{3-(cis-2-Azabicyclo[3.1.0]hex-2-yl)propoxy}-N-methylbenzamide hydrochloride

Step 1: 4-[3-(cis-2-Azabicyclo[3.1.0]hex-2-yl)propoxy]-N-methylbenzamide

The test procedure is the same as that of Example 1, synthesis route A, Steps 1 and 2, replacing the 4-hydroxybenzamide in Step 1 by 4-hydroxy-N-methylbenzamide.

Elemental Microanalysis:

| | % C | % H | % N |
|---|---|---|---|
| Calculated | 70.04 | 8.08 | 10.21 |
| Found | 69.57 | 8.04 | 10.17 |

Step 2: 4-[3-(cis-2-Azabicyclo[3.1.0]hex-2-yl)propoxy]-N-methylbenzamide hydrochloride

The test procedure is the same as that of Example 1, synthesis route A, Step 3.

### EXAMPLE 7a

### 4-{3-(cis-2-Azabicyclo[3.1.0]hex-2-yl)propoxy}-N-methylbenzamide hydrochloride (enantiomer 1)

Enantiomer 1 was obtained by preparative separation on a chiral column, CHIRALPACK IA 20 μm loaded at 0.3 g/650 g, eluant mixture: acetonitrile/diethylamine (100/0.1), flow rate 100 ml/min, UV detection at 280 nm.

Optical rotation: [α_{D}]_{589 nm}^{22°}=−58.06° (c=1.0; MeOH)

### EXAMPLE 7b

### 4-{3-(cis-2-Azabicyclo[3.1.0]hex-2-yl)propoxy}-N-methylbenzamide hydrochloride (enantiomer 2)

Enantiomer 2 was obtained by preparative separation on a chiral column, CHIRALPACK IA 20 μm loaded at 0.3 g/650 g, eluant mixture: acetonitrile/diethylamine (100/0.1), flow rate 100 ml/min, UV detection at 280 nm.

Optical rotation: [α_{D}]_{589 nm}^{22°}=+58.81° (c=1.0; MeOH)

### EXAMPLE 8

### N-(4-{3-(cis-2-azabicyclo[3.1.0]hex-2-yl)propoxy}phenyl)-N-methyl-acetamide hydrochloride

The test procedure is the same as that of Example 1, synthesis route A, replacing the 4-hydroxybenzamide in Step 1 by N-(4-hydroxyphenyl)-N-methylacetamide.

Elemental Microanalysis:

| | % C | % H | % N | % Cl− |
|---|---|---|---|---|
| Calculated | 62.86 | 7.76 | 8.62 | 10.91 |
| Found | 62.08 | 7.12 | 8.48 | 11.02 |

### EXAMPLE 8a

### N-(4-{3-(cis-2-azabicyclo[3.1.0]hex-2-yl)propoxy}phenyl)-N-methyl-acetamide hydrochloride

Enantiomer 1 was obtained by preparative separation on a chiral column, CHIRALPACK IA 20 μm loaded at 0.5 g/650 g, eluant mixture: acetonitrile/diethylamine (100/0.1), flow rate 100 ml/min, UV detection at 295 nm.

Optical rotation: [α_{D}]_{589 nm}^{22°}=−23.52° (c=1.02; MeOH)

### EXAMPLE 8b

### N-(4-{3-(cis-2-azabicyclo[3.1.0]hex-2-yl)propoxy}phenyl)-N-methyl-acetamide hydrochloride

Enantiomer 2 was obtained by preparative separation on a chiral column, CHIRALPACK IA 20 μm loaded at 0.5 g/650 g, eluant mixture: acetonitrile/diethylamine (100/0.1), flow rate 100 ml/min, UV detection at 295 nm.

Optical rotation: [α_{D}]_{589 nm}^{22°}=+24.17° (c=1.0; MeOH)

### EXAMPLE 9

### N-(4-{3-(cis-2-azabicyclo[3.1.0]hex-2-yl)propoxy}phenyl)-2-methoxy-acetamide hydrochloride

The test procedure is the same as that of Example 1, synthesis route A, replacing the 4-hydroxybenzamide in Step 1 by N-(4-hydroxyphenyl)-2-methoxyacetamide.

Elemental Microanalysis:

| | % C | % H | % N | % Cl− |
|---|---|---|---|---|
| Calculated | 60.92 | 7.67 | 7.89 | 9.99 |
| Found | 59.91 | 7.63 | 7.76 | 9.65 |

### Pharmacological Study

### EXAMPLE A

### Cerebral Levels of N′-methylhistamine in the NMRI Mouse

The purpose of this study, which was carried out in accordance with the method of Taylor et al. (Biochem. Pharm., 1992, 44, 1261-1267), is to evaluate the ex vivo activity of the compounds of the present invention as antagonists of type H₃ central histamine receptors. That activity is revealed by measuring, after treatment with the test compounds by the oral route, the central levels of N⁹⁶ -methylhistamine, which is a main metabolite of histamine. An increase in the cerebral concentrations of N^{τ}-methylhistamine indicates an increase in to the turn-over of histamine by blockage of the type H₃ central histamine receptors.

NMRI mice (18-20 g) are treated with compounds of the present invention or with their carrier (20 ml/kg) by the oral route. One hour after the pharmacological treatment, the animals are sacrificed; the brains are removed, frozen in liquid nitrogen, weighed and homogenised in 0.1N HClO₄ at 4° C. The homogenised products are centrifuged (15000 g, 17 mins., 4° C.). The supernatants are recovered and divided into aliquots. The aliquots are frozen in liquid nitrogen and stored at −80° C. until they are analysed.

Determination of the cerebral levels of N^{τ}-methylhistamine is carried out by capillary electrophoresis. The tissue levels of N^{τ}-methylhistamine are expressed in μg/g of fresh brain. The comparison of the cerebral levels of N^{τ}-methylhistamine between animals treated with the carrier (controls) and animals treated with compounds of the present invention is carried out by single-factor variance analysis followed, if necessary, by a complementary analysis (Dunnett's test).

The results show that, at a dose of 3 mg/kg PO, the compounds of the present invention are capable of significantly increasing endogenous cerebral concentrations of N^{τ}-methyl-histamine by more than 200%.

By way of example, the compounds of Examples 4, 9, 8, 7, 6 and 3, when administered at 3 mg/kg PO, increase the endogenous cerebral concentrations of N^{τ}-methylhistamine respectively by:
Compound of Example 4: +221%
Compound of Example 9: +250%
Compound of Example 8: +276%
Compound of Example 7: +377%
Compound of Example 6: +225%
Compound of Example 3: +272%

These results demonstrate that the compounds of the present invention are powerful antagonists of type H₃ central histamine receptors.

### EXAMPLE B

### Affinity for Mouse H₃ Receptors

The purpose is to measure the affinity of compounds of the present invention for type H₃ mouse histamine receptors transfected into CHO cells.

The compounds are incubated at different concentrations in the presence of transfected CHO cells, iodoproxyfan as a radiolabelled ligand which is specific for H₃ receptors, and scintillant beads for 24 h at room temperature.

At the end of the incubation, the displacement of the specific binding of the ligand by the compounds under test is measured, and the affinity constants of those compounds for mouse H₃ receptors are determined

The results show that the compounds of the invention have affinity for type H₃ histamine receptors. For instance:
Compound of Example 1: Ki=2.4 μM
Compound of Example 3: Ki=0.75 μM
Compound of Example 5: Ki=0.18 μM
Compound of Example 9: Ki=0.39 μM

### EXAMPLE C

### Pharmaceutical Compositions

Formula for the preparation of 1000 tablets each containing 100 mg of active ingredient:

| | |
|---|---|
| Compound of Example 4 | 100 g |
| Hydroxypropylcellulose | 20 g |
| Polyvinylpyrrolidone | 20 g |
| Wheat starch | 150 g |
| Lactose | 900 g |
| Magnesium stearate | 30 g |

1-24. (canceled)

25. A compound selected from those of formula (I): 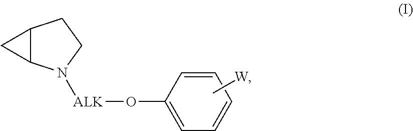
wherein:
ALK represents an alkylene chain,
W represents a group 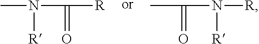
wherein R and R′, each independently of the other, represent a hydrogen atom or a linear or branched (C₁-C₆)alkyl group optionally substituted by one or more groups selected from halogen, hydroxy and alkoxy,
it being understood that:
the term “alkylene” denotes a linear or branched divalent radical having from 2 to 6 carbon atoms,
the term “alkoxy” denotes an alkyl-oxy group in which the alkyl chain, which is linear or branched, has from 1 to 6 carbon atoms,
its enantiomers and diastereoisomers, and addition salts thereof with a pharmaceutically acceptable acid or base.

26. The compound of claim 25, wherein the W group is located in the para position.

27. The compound of claim 25, wherein ALK represents an ethylene or propylene group.

28. The compound of claim 25, wherein ALK represents a propylene group.

29. The compound of claim 25, wherein W represents

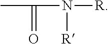

30. The compound of claim 25, wherein W represents

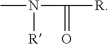

31. The compound of claim 25, wherein R and R′, each independently of the other, represent a hydrogen atom, a methyl group or an ethyl group, wherein the methyl group and the ethyl group may be optionally substituted by a methoxy group.

32. The compound of claim 25, wherein W represents —CO—NH₂, —CO—NH—CH₃, —CO—N(CH₃)₂, —CO—N(CH₂CH₃)₂, —NH—CO—CH₃, —N(CH₃)—CO—CH₃ or —NH—CO—CH₂—OCH₃.

33. The compound of claim 25, which is selected from:
4-{3-(cis-2-azabicyclo[3.1.0]hex-2-yl)propoxy}-N,N-dimethylbenzamide,
4-{3-(cis-2-azabicyclo[3.1.0]hex-2-yl)propoxy}-N,N-diethylbenzamide,
N-(4-{3-(cis-2-azabicyclo[3.1.0]hex-2-yl)propoxy}phenyl)-N-methylacetamide,
4-[3-(cis-2-azabicyclo[3.1.0]hex-2-yl)propoxy]benzamide,
N-(4-{3-(cis-2-azabicyclo[3.1.0]hex-2-yl)propoxy}phenyl)acetamide,
4-{3-(cis-2-azabicyclo[3.1.0]hex-2-yl)propoxy}-N-methylbenzamide,
N-(4-{3-(cis-2-azabicyclo[3.1.0]hex-2-yl)propoxy}phenyl)-2-methoxyacetamide,
N-(4-{2-(cis-2-azabicyclo[3.1.0]hex-2-yl)ethoxy}phenyl)acetamide,
and enantiomers, and addition salts thereof with a pharmaceutically acceptable acid or base.

34. A compound selected from those of formula (VI): 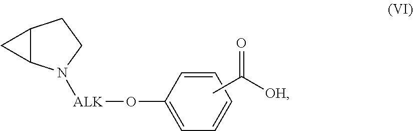
wherein ALK represents an alkylene chain having from 2 to 6 carbon atoms.

35. A compound selected from those of formula (VII): 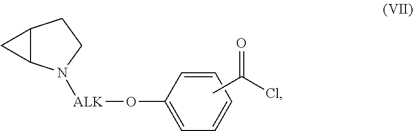
wherein ALK represents an alkylene chain having from 2 to 6 carbon atoms.

36. A compound selected from those of formula (VIII): 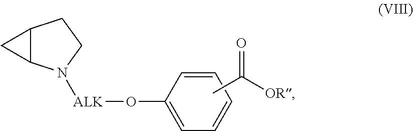
wherein ALK represents an alkylene chain having from 2 to 6 carbon atoms and R″ is a linear or branched (C₁-C₆)alkyl group or a benzyl group.

37. A compound selected from those of formula (IX): 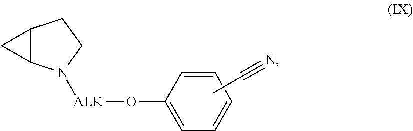
wherein ALK represents an alkylene chain having from 2 to 6 carbon atoms.

38. A pharmaceutical compositions comprising as active ingredient a compound of claim 25, in combination with one or more pharmaceutically acceptable excipients.

39. A method of treating a condition selected from cognitive and psycho-behavioural disorders associated with cerebral ageing, with neurodegenerative diseases or with cranial traumas, in a subject in need thereof, comprising administration of an effective amount of a compound of claim 25.

40. The method of claim 39, wherein the condition is selected from cognitive and psycho-behavioural disorders associated with Alzheimer's disease, Parkinson's disease, Pick's disease, Lewy body dementias, frontal and subcortical dementias, frontotemporal dementias, vascular dementias, Huntington's disease and multiple sclerosis.

41. The method of claim 39, wherein the psycho-behavioural disorder is selected from sleep disorders, apathy and anxio-depressive states.

42. The method of claim 40, wherein the condition is selected from sleep disorders associated with Alzheimer's disease and with Parkinson's disease.

43. A method of treating a condition selected from mood disorders, anxio-depressive states, Tourette's syndrome, schizophrenia and cognitive disorders associated therewith, pain, sleep disorders, sleep-waking rhythm disorders, and attention-deficit hyperactivity syndrome, in a subject in need thereof, comprising administration of an effective amount of a compound of claim 25.

44. The method of claim 43, wherein the condition is a sleep disorder selected from narcolepsy, hypersomnia occurring in obstructive sleep apnoea syndrome or in attention-deficit hyperactivity syndrome, and diurnal somnolence.

45. A composition comprising a compound of claim 25 in combination with an acetylcholinesterase inhibitor.

46. A composition comprising a compound of claim 25 in combination with donepezil, galantamine or rivastigmine.

47. A method of treating cognitive disorders associated with Alzheimer's disease, in a subject in need thereof, comprising administration of an effective amount of the composition of claim 45.
